(19) United States
(12) Plant Patent Application Publication
(10) Pub. No.: US 2019/0373792 P1
(43) Pub. Date: Dec. 5, 2019

(54) Haworthia hybrid plant named 'AMIHAW1811'

(50) ;

(71) Applicant: NovoAmi B.V., Heerhugowaard (NL)

(72) Inventor: Gerard van Langen, Heerhugowaard (NL)

(73) Assignee: NovoAmi B.V., Heerhugowaard (NL)

(21) Appl. No.: 16/501,724

(22) Filed: May 29, 2019

(30) Foreign Application Priority Data
May 29, 2018 (QZ) 2018/1401

(51) Int. Cl. A01H 6/00

(52) U.S. Cl. PLT/373

(57) ABSTRACT

A new and distinct Haworthia hybrid plant named ‘AMIHAW1811’, which is characterized by spirally-arranged, dark green and glossy foliage with near-white orbicular protuberances arranged in irregular transverse rows across the upper and lower leaf surfaces and small near-white teeth along the length of the margins. The new variety has shown to be uniform and stable in the resulting generations from asexual propagation.

### CROSS REFERENCE TO RELATED APPLICATIONS

This application claims priority to the Community Plant Variety Rights application number 2018/1401, filed May 29, 2018, which is herein incorporated by reference.

Latin name of the genus and species: The Latin name of the genus and species of the novel variety disclosed herein is Haworthia limifolia x Gasteria sp.

Variety denomination: The inventive variety of Haworthia hybrid disclosed herein has been given the variety denomination ‘AMIHAW1811’.

### BACKGROUND OF THE INVENTION

Parentage: ‘AMIHAW1811’ is a seedling selection resulting from the controlled pollination of an unnamed Haworthia limifolia plant (not patented), the seed parent, with Gasteria hybrid ‘Green Star’ (U.S. Plant Pat. No. 24,763), the pollen parent. The crossing was made by the inventor in the winter of 2014 at a commercial greenhouse in Heerhugowaard, the Netherlands. In the summer of 2015, one seedling was observed which exhibited unique growth and foliage characteristics. After confirming the stability of the unique characteristics first observed, the new plant was selected for commercialization and given the name, ‘AMIHAW1811’.

Asexual Reproduction: Asexual reproduction of the new cultivar ‘AMIHAW1811’, by way of rooting leaf cuttings, was first initiated in the summer of 2016 at the inventor's commercial greenhouse in Heerhugowaard, the Netherlands. Through four subsequent generations, the unique features of this cultivar have proven to be stable and true to type.

### SUMMARY OF THE INVENTION

The cultivar ‘AMIHAW1811’ has not been observed under all possible environmental conditions. The phenotype may vary somewhat with variations in environment such as temperature, day length, and light intensity, without, however, any variance in genotype. The following traits have been repeatedly observed and are determined to be the unique characteristics of ‘AMIHAW1811’. These characteristics in combination distinguish ‘AMIHAW1811’ as a new and distinct Haworthia hybrid cultivar:
1. ‘AMIHAW1811’ exhibits spirally-arranged succulent foliage in a basal rosette; and
2. ‘AMIHAW1811’ exhibits relatively small and thick foliage; and
3. ‘AMIHAW1811’ exhibits foliage with small, near-white teeth along the length of the margins and distal portion of the abaxial midrib; and
4. ‘AMIHAW1811’ exhibits dark green, moderately glossy foliage with near-white orbicular protuberances densely covering the abaxial and adaxial surfaces.

### BRIEF DESCRIPTION OF THE FIGURES

FIG. 1 illustrates, as nearly true as it is reasonably possible to make the same in color photographs of this type, an exemplary plant of ‘AMIHAW1811’ grown in a commercial greenhouse in Heerhugowaard, the Netherlands. This plant is approximately 1 year old, shown planted in a 11 cm container.

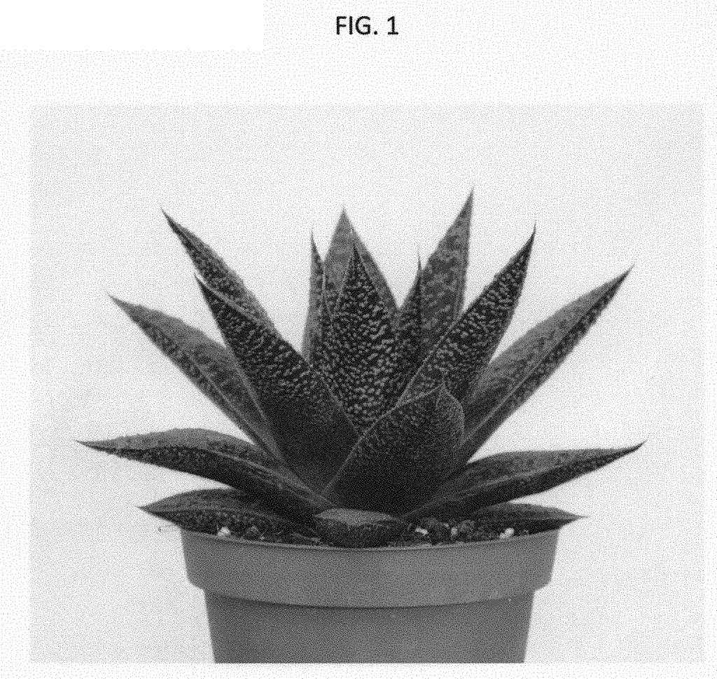

FIG. 2 illustrates, as nearly true as it is reasonably possible to make the same in color photographs of this type, the typical foliage arrangement of ‘AMIHAW1811.

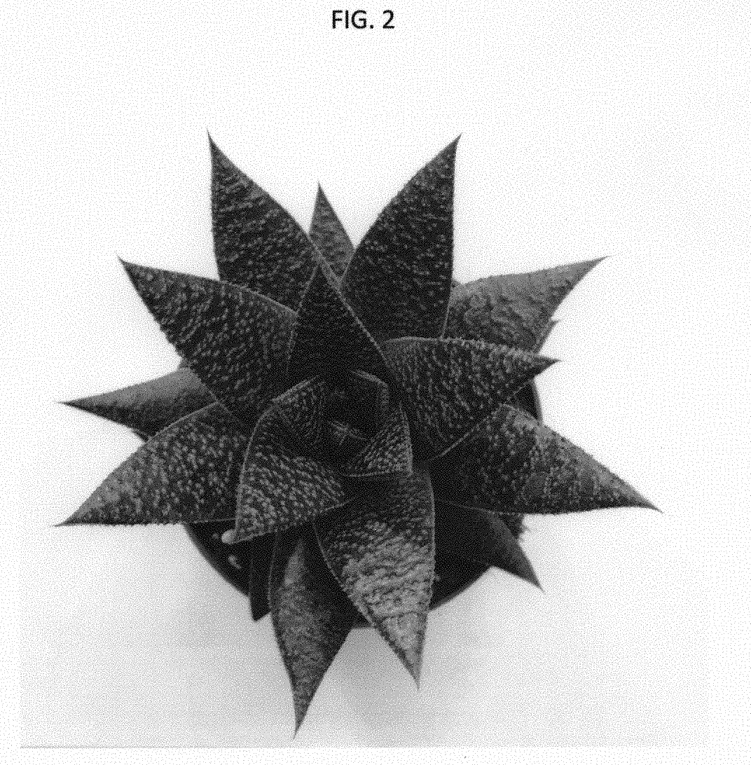

FIG. 3 illustrates, as nearly true as it is reasonably possible to make the same in color photographs of this type, the adaxial surface of the mature foliage ‘AMIHAW1811’.

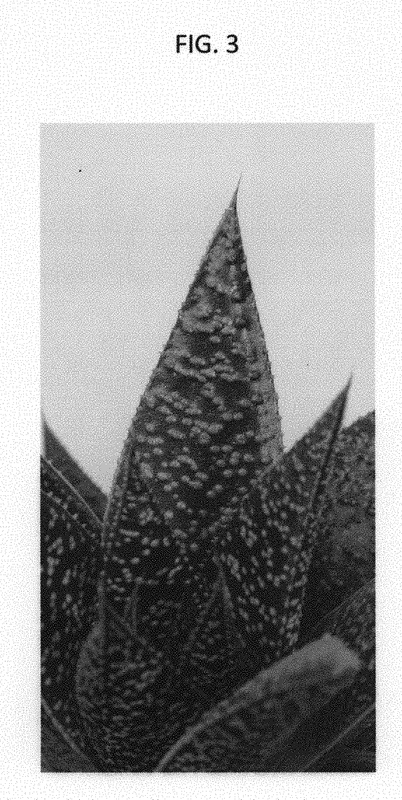

FIG. 4 illustrates, as nearly true as it is reasonably possible to make the same in color photographs of this type, the abaxial surface of the mature foliage ‘AMIHAW1811’.

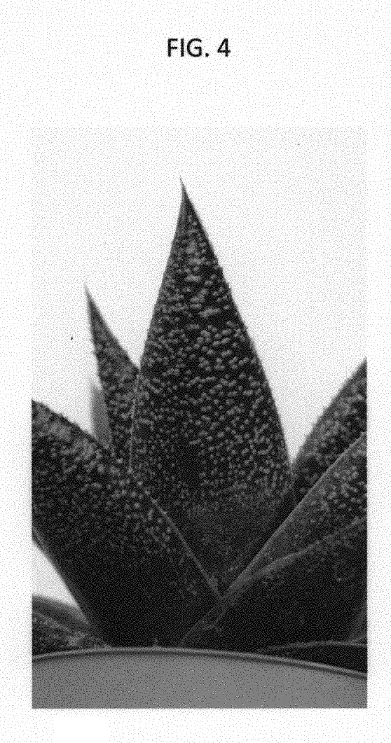

### BOTANICAL DESCRIPTION OF THE PLANT

The following observations and measurements made in April of 2019 describe averages from a sample set of six specimens of 1 year-old ‘AMIHAW1811’ plants grown in 11 cm nursery containers at commercial greenhouse in Heerhugowaard, the Netherlands. Plants were produced using conventional greenhouse production protocols for Haworthia plants which consisted of minimal irrigation and fertilizer applications, and chemical pest and disease control measures against mealy bug and Botrytis as required. Plants were grown under approximately 50 percent shade after propagation and later exposed to full sun once they began to mature. No photoperiodic treatments or artificial light was given to the plants.

Those skilled in the art will appreciate that certain characteristics will vary with older or, conversely, with younger plants. ‘AMIHAW1811’ has not been observed under all possible environmental conditions. Where dimensions, sizes, colors and other characteristics are given, it is to be understood that such characteristics are approximations or averages set forth as accurately as practicable. The phenotype of the variety may differ from the descriptions set forth herein with variations in environmental, climatic and cultural conditions. Color notations are based on The Royal Horticultural Society Colour Chart, The Royal Horticultural Society, London, 2015 (sixth edition).

A botanical description of ‘AMIHAW1811’ and a comparison with the parents and closest known comparator is provided below.
Plant description:
   Growth habit.—Succulent perennial with foliage growing in a stemless, non-branched basal rosette.
   Plant shape.—Flattened globular.
   Height from soil level to top of foliar plane.—9.3 cm.
   Plant spread.—Average of 15.9 cm.
   Growth rate.—Slow to moderately fast.
   Plant vigor.—Moderately vigorous.
   Propagation.—Type — Leaf cuttings. Time to initiate rooting — Approximately 21 days at 18 degrees Celsius. Crop time — Approximately 35 weeks to produce a marketable plant in a 10.5 cm container.
   Disease and pest resistance or susceptibility.—Neither resistance nor susceptibility to typical Haworthia or Gasteria pests and diseases has been observed.
   Environmental tolerances.—Adapt to, at least, USDA Zones 10 to 12 and temperatures as high as 40 degrees Celsius; low tolerance to rain; high tolerance to wind.
Root System:
   General.—Fine, well-branched fibrous roots.
Stems:
   Branching habit.—Leaves in a non-branching basal rosettes; no main branches or lateral branches present.
Foliage:
   Arrangement.—Spirally arranged in a rosette.
   Division.—Simple.
   Attachment.—Sessile.
   Quantity.—24 leaves per rosette.
   Shape.—Ovate, succulent foliage.
   Dimensions.—9.2 cm long, 3.3 cm wide, and 1.1 cm thick, on average.
   Aspect.—Slightly concaved and carinate.
   Attitude.—Juvenile foliage at the center of the rosette is held upward to slightly outward; foliage becomes progressively more relaxed towards the outer whorls of mature foliage, at an average angle of 40 degrees from horizontal.
   Apex.—Apiculate, with a soft mucronate tip.
   Base.—Broad cuneate.
   Margin.—Finely dentate; teeth have an average length of 0.1 cm and are colored green-white, nearest to RHS 157D. Margins are not undulated or lobed.
   Texture, adaxial surface.—Glabrous and densely covered with orbicular protuberances; protuberances are approximately 0.1 cm high and 0.1 cm in diameter, at maturity.
   Texture, abaxial surface.—Glabrous and densely covered with orbicular protuberances; protuberances are approximately 0.1 cm high and 0.1 cm in diameter, at maturity. The distal one-third portion of the midrib is finely dentate; teeth have an average length of 0.75 mm and are colored green-white, nearest to RHS 157A.
   Luster, adaxial surface.—Moderately glossy.
   Luster, abaxial surface.—Moderately glossy.
   Color.—Juvenile foliage, adaxial surface — Nearest to in between green and yellow-green, RHS NN137A and 147A, and fading to yellow-green towards the base, nearest to RHS 148D; protuberances are colored yellow-green, nearest to RHS 145B. Juvenile foliage, abaxial surface — Green, nearest to RHS NN137A, and fading to yellow-green towards the base, nearest to RHS 148D; protuberances are colored yellow-green, nearest to RHS 147D. Dentate teeth on the midrib are green-white, nearest to RHS 157A. Mature foliage, adaxial surface — Nearest to in between yellow-green and greyed-green, RHS 147A and N189A; fading to greyed-white towards the base, nearest to a mixture of RHS 156B and 156C; protuberances are colored yellow-green, nearest to RHS 146C. Mature foliage, abaxial surface — Yellow-green, nearest to RHS 147A, and fading to a mixture of greyed-white and greyed-orange, RHS 156B and N170D; protuberances are colored yellow-green, nearest to RHS 146B. Dentate teeth on the midrib are green-white, nearest to RHS 157A.
   Venation.—No venation is visible.
   Petiole.—No petiole; leaves are sessile.
Inflorescence: No flowering has been observed to date.
Comparisons with the parent plant and closest known comparator: Plants of the new cultivar ‘AMIHAW1811’ differ from the seed parent, an unnamed Haworthia limifolia (not patented), in the following characteristics described in Table 1 below.

TABLE 1

| | | Haworthia limifolia seed |
|---|---|---|
| Characteristic | ‘AMIHAW1811’ | parent |
| Rate of growth. | Faster than the seed | Slower than |
| | parent. | ‘AMIHAW1811’. |
| Plant size. | Larger than the seed | Smaller than |
| | parent. | ‘AMIHAW1811’. |
| General coloration | Dark green. | Greyed-green. |
| of the mature | | |
| foliage. | | |
| Foliage length. | Longer than the seed | Shorter than |
| | parent. | ‘AMIHAW1811’. |
| Leaf protuberances. | Orbicular protuberances | Conspicuous transverse |
| | densely cover the leaf | linear protuberances |
| | surface; protuberances | presenting as raised ribs |
| | are randomly placed. | across the leaf surface. |
| General coloration | Lighter green when | Nearly the same color |
| of leaf | compared to the rest | green as the leaf surface. |
| protuberances. | of the leaf surface. | |

Plants of the new cultivar ‘AMIHAW1811’ differ from the pollen parent, Gasteria hybrid ‘Green Star’ (U.S. Plant Pat. No. 24,763), in the following characteristics described in Table 2 below.

TABLE 2

| Characteristic | ‘AMIHAW1811’ | ‘Green Star’ |
|---|---|---|
| Foliage attitude. | More upright than | More relaxed than |
| | ‘Green Star’. | ‘AMIHAW1811’. |
| Foliage thickness. | Thicker than | Thinner than |
| | ‘Green Star’. | ‘AMIHAW1811’. |
| General coloration | Darker shade of green | Lighter shade of green |
| of the mature foliage. | than ‘Green Star’. | than ‘AMIHAW1811’. |

Comparisons with the closest known comparator: Plants of the new cultivar ‘AMIHAW1811’ differ from the closest known commercial comparator, the common form of the species Gasteria sp. ‘Flow’ (not patented), in the following characteristics described in Table 3 below.

TABLE 3

| Characteristic | ‘AMIHAW1811’ | ‘Flow’ |
|---|---|---|
| General coloration of | Darker shade of green | Lighter shade of green |
| the mature foliage. | than ‘Flow’. | than ‘AMIHAW1811’. |
| Foliage length. | Longer than ‘Flow’. | Shorter than |
| | | ‘AMIHAW18111’. |
| Foliage thickness. | Thicker than ‘Flow’. | Thinner than |
| | | ‘AMIHAW1811’. |
| Prominence of leaf | More prominent. | Less prominent. |
| protuberances. | | |

That which is claimed is:

1. A new and distinct variety of Haworthia hybrid plant named ‘AMIHAW1811’, substantially as described and illustrated herein.
